(19) United States
(12) Patent Application Publication
(10) Pub. No.: US 2022/0240488 A1
(43) Pub. Date: Aug. 4, 2022

(54) PET DIAPER AND MANUFACTURING METHOD OF PET DIAPER

(71) Applicant: Unicharm Corporation, Ehime (JP)

(72) Inventor: Daisuke Komatsubara, Kanonji-shi (JP)

(21) Appl. No.: 17/612,749

(22) PCT Filed: May 19, 2020
(86) PCT No.: PCT/JP2020/019766

§ 371 Date: Nov. 19, 2021

(30) Foreign Application Priority Data
May 20, 2019 (JP) 2019-094865

(51) Int. Cl. A01K 23/00; A61F 13/84

(52) U.S. Cl. CPC A01K 23/00; A61F 2013/8497; A61F 13/84

(57) ABSTRACT

To provide a pet diaper which can be worn in a suitable position and which can suppress leakage of excrement. [Solution] This pet diaper (1) comprises: a waste circumferential direction (W) which is arranged along the waist circumference of the pet; a crossing direction (Z) which is perpendicular to the waist circumference direction; a main body (2) which has a top sheet (10), a back sheet (20) and an absorbent core (30) arranged between the top sheet and the back sheet; and a joining part (40) which joins the back sheet side of the main body and the front sheet side of the main body. The absorbent core is arranged to the inside of the two ends of the main body in the waist circumference direction. This pet diaper has a guide part (50) which guides the wearing position of the pet diaper.

### FIELD

The present invention relates to a pet diaper for pets, such as dogs and cats, to wear.

### BACKGROUND

Patent Literatures 1 and 2 disclose pet diapers. The pet diaper of Patent Literature 1 includes a belly pad portion brought into contact with the belly side of a pet, a back pad portion brought into contact with the back side of the pet, a tail hole which is provided in the back pad portion and through which the tail passes, and a fastener tape provided in the belly pad portion. When the pet diaper of Patent Literature 1 is put on a pet, the tail hole is slipped over the tail, the belly pad portion is brought into contact with the belly side of the pet, the back pad portion is brought into contact with the back side, and the fastener tape is fastened to the back pad portion (see FIG. 4 of Patent Literature 1).

The pet diaper of Patent Literature 2 includes a band-shaped body having an absorbent core, an adhesive seal arranged on the top surface side of the body, and a seal receiving portion arranged on the back surface side of the body. When the pet diaper of Patent Literature 2 is put on a pet, the waistline direction of the body is aligned with the waistline direction of the pet, the pet diaper is positioned so that the pet's urethral orifice is covered with the absorbent core, and the pet diaper is wrapped around the pet's waist and fastened through the adhesive seal and the seal receiving portion (see FIG. 3 of Patent Literature 2).

### CITATION LIST

### Patent Literature

Patent Literature 1: Japanese Unexamined Patent Publication No. 2003-210062
Patent Literature 2: Japanese Utility Model Registration No. 3141580

### SUMMARY

The pet diapers of Patent Literatures 1 and 2 have the following problems.

When the pet diaper is put on a pet, the pet to wear the diaper does not always stand still. Therefore, if the pet moves, sometimes it is impossible to put the pet diaper on a proper position. Furthermore, because the pet's shape, such as waist size, varies with the type of pet and the seasons, sometimes the pet diaper cannot be put on a proper position. In addition, even if the pet diaper is put on a position deviating from the proper position, sometimes the user cannot notice such a problem based on the pet's response and cannot properly correct the position.

Generally, in a pet diaper, the absorbent core is not arranged to fully wrap up the pet's waist, and is arranged only in a region absorbing excrement, that is, in a part of the waist. Therefore, in a case where the pet diaper is not put on a proper position, there is a risk that excrement will leak.

An aspect of the present invention is to provide a pet diaper which can be put on a proper position and can inhibit the leakage of excrement.

A pet diaper according to one aspect has a waistline direction arranged along a waistline direction of a pet and a cross direction orthogonal to the waistline direction, the pet diaper comprising a main-body portion that has a topsheet, a backsheet, and an absorbent core arranged between the topsheet and the backsheet, and a joining portion that joins a backsheet-side surface of the main-body portion and a topsheet-side surface of the main-body portion. The absorbent core is arranged away from two end edges of the main-body portion toward an inner side in the waistline direction, and the pet diaper has a marked portion as a guide showing where the pet diaper is to be put on.

### BRIEF DESCRIPTION OF DRAWINGS

FIG. 1 is a plan view of a pet diaper according to a first embodiment, seen from a skin facing side.

FIG. 2 is a plan view of a pet diaper according to the first embodiment, seen from a non-skin facing side.

FIG. 3 is a schematic cross-sectional view of the pet diaper taken along a line A-A shown in FIG. 1.

FIG. 4 is a view schematically showing the pet diaper according to the first embodiment that is worn by a pet.

FIG. 5 is a plan view of a pet diaper according to a second embodiment, seen from a skin facing side.

FIG. 6 is a plan view of the pet diaper according to the second embodiment, seen from a non-skin facing side.

FIG. 7 is a view schematically showing the pet diaper according to the second embodiment that is worn by a pet.

FIG. 8 is a plan view of a pet diaper according to a third embodiment, seen from a non-skin facing side.

FIG. 9 is a plan view of a pet diaper according to a fourth embodiment, seen from a non-skin facing side.

FIG. 10 is a plan view of a pet diaper according to a fifth embodiment, seen from a non-skin facing side.

### DESCRIPTION OF EMBODIMENTS

### (1) Summary of Embodiment

At least following matters will become clear with description of this specification and attached drawings.

A pet diaper according to one aspect has a waistline direction arranged along a waistline direction of a pet and a cross direction orthogonal to the waistline direction, the pet diaper comprising a main-body portion that has a topsheet, a backsheet, and an absorbent core arranged between the topsheet and the backsheet, and a joining portion that joins a backsheet-side surface of the main-body portion and a topsheet-side surface of the main-body portion. The absorbent core is arranged away from two end edges of the main-body portion toward an inner side in the waistline direction, and the pet diaper has a marked portion as a guide showing where the pet diaper is to be put on. According to the present aspect, the user can position the pet diaper based on the marked portion. Therefore, the user can put the pet diaper on a proper position. Furthermore, even though the user cannot put the pet diaper on a proper position or the pet diaper is displaced after being worn, the user can notice the displacement or twisting of the diaper based on the marked portion and can properly correct the position or adjust the diaper to an appropriate state. Therefore, in a pet diaper in which the absorbent core is shorter than the main-body portion in the waistline direction, the absorbent core is arranged at an appropriate position where the absorbent core can absorb excrement, which makes it possible to inhibit the leakage of excrement.

According to a preferable aspect, the marked portion may be configured to be visible from the topsheet side of the pet diaper. The topsheet side of the pet diaper is the side of a surface brought into contact with a pet when the diaper is put on the pet. The user can position the pet diaper while bringing the marked portion into contact with the pet's body.

According to a preferable aspect, the marked portion may be configured to be visible from the backsheet side of the pet diaper. The backsheet side of the pet diaper is positioned on the outer side when the diaper is put on a pet, and remains visible even after the pet wears the diaper. Therefore, the user can position the diaper not only in the process of putting the diaper on a pet but also after putting the diaper on a pet. Even though the diaper turns in the waistline direction and is displaced when put on a pet, the user can notice the displacement based on the marked portion and can properly correct the position.

According to a preferable aspect, the marked portion may have a waist center marked portion that indicates a center of the absorbent core in the waistline direction. According to the present aspect, in a pet diaper in which the absorbent core is shorter than the waistline, the absorbent core can be arranged at a position where the absorbent core can absorb excrement, which makes it possible to inhibit the leakage of excrement.

According to a preferable aspect, a plurality of marked portions may be arranged in the waistline direction by being spaced apart from each other. According to the present aspect, a plurality of marked portions are arranged along the waistline direction. Therefore, the user can position the diaper based on the marked portions in various directions (for example, by seeing the marked portions from the right side or the left side). Furthermore, the user can notice the displacement occurring in various directions, such as the displacement or twisting on the right side and the displacement or twisting on the left side, and can adjust the diaper to an appropriate state.

According to a preferable aspect, the marked portion may have a pair of waist side marked portions that are arranged on two sides facing each other across the center of the absorbent core in the waistline direction. According to the present aspect, by the waist side marked portions that are arranged on two sides facing each other across the center of the absorbent core in the waistline direction, the user can easily ascertain the position of the absorbent core in the waistline direction. Therefore, the user can appropriately arrange the absorbent core in the waistline direction, which makes it possible to inhibit the leakage of excrement.

According to a preferable aspect, one of the pair of waist side marked portions may be arranged in a region that overlaps one end portion of the absorbent core in the waistline direction, and the other may be arranged in a region that overlaps the other end portion of the absorbent core in the waistline direction. According to the present aspect, the user can ascertain the positions of two end portions of the absorbent core in the waistline direction and can arrange the absorbent core so that leakage does not occur.

According to a preferable aspect, one of the pair of waist side marked portions may be arranged away from one end edge of the absorbent core in the waistline direction toward the outer side in the waistline direction, and the other may be arranged away from the other end edge of the absorbent core in the waistline direction toward the outer side in the waistline direction. According to the present aspect, by the marked portion arranged on the outer side in the waistline direction, the user can ascertain the position of the region where the absorbent core is not arranged and can arrange the absorbent core so that leakage does not occur.

According to a preferable aspect, a stretchable member that stretches and contracts in at least one of the waistline direction and the cross direction may be arranged in the main-body portion, and the marked portion may include a stretching marked portion arranged in a region that overlaps the stretchable member. According to the present aspect, the stretchable member stretches and contracts in at least one of the waistline direction and the cross direction, and is put on a pet so as to be in close contact with the pet's body. Because the marked portion is arranged in the region that overlaps the stretchable member, the user can ascertain the position of the stretchable member that is in close contact with the pet's body and can arrange the stretchable member at an appropriate position.

According to a preferable aspect, the absorbent core may be arranged away from two end edges of the main-body portion toward the inner side in the cross direction, and the marked portion may have a cross center marked portion that indicates the center of the absorbent core in the cross direction. According to the present aspect, in a pet diaper in which the absorbent core is short in the cross direction, the absorbent core can be arranged at a position where the absorbent core can absorb excrement, which makes it possible to inhibit the leakage of excrement.

According to a preferable aspect, the marked portion may be a plurality of marked portions arranged in the cross direction by being spaced apart from each other. According to the present aspect, the plurality of marked portions is arranged in the cross direction. Therefore, the user can position the diaper based on the marked portions in various directions (for example, by seeing the marked portions from the front side or the rear side).

According to a preferable aspect, the marked portion may have a pair of cross side marked portions on arranged in the cross direction, on two sides facing each other across the center of the absorbent core in the cross direction. According to the present aspect, by the cross side marked portions arranged on two sides facing each other across the center of the absorbent core in the cross direction, the user can easily ascertain the position of the absorbent core in the cross direction. Therefore, the user can appropriately arrange the absorbent core in the cross direction, which makes it possible to inhibit the leakage of excrement.

According to a preferable aspect, one of the pair of cross side marked portions may be arranged in a region that overlaps one end portion of the absorbent core in the cross direction, and the other may be arranged in a region that overlaps the other end portion of the absorbent core in the cross direction. According to the present aspect, the user can ascertain the positions of two end portions of the absorbent core in the cross direction, and can arrange the absorbent core so that leakage does not occur.

According to a preferable aspect, one of the pair of cross side marked portions may be arranged away from one end edge of the absorbent core in the cross direction toward the outer side in the cross direction, and the other may be arranged away from the other end edge of the absorbent core in the cross direction toward the outer side in the cross direction. According to the present aspect, the user can ascertain the position of the region where the absorbent core is not arranged, and can arrange the absorbent core so that leakage does not occur.

According to a preferable aspect, a first decorative portion and a second decorative portion different from the first decorative portion in design may be printed on the backsheet, and the marked portion may have a boundary marked portion configured with the boundary between the first decorative portion and the second decorative portion. The present aspect makes it possible to position the diaper based on the marked portion while improving decorativeness by the designs of the two portions, the first decorative portion and the second decorative portion.

According to a preferable aspect, the marked portion may have an end edge marked portion that indicates the outer end edge of the main-body portion in the cross direction. According to the present aspect, the outer end edge of the main-body portion in the cross direction can be appropriately arranged, and the entire main-body portion can be put on a pet by being appropriately wrapped around the pet. Therefore, the diaper can fit better the pet, and discomfort resulting from excessive tightness can be reduced.

A pet diaper according to another aspect has a waistline direction arranged along a waistline direction of a pet and a cross direction orthogonal to the waistline direction, the pet diaper comprising a main-body portion that has a topsheet, a backsheet, and an absorbent core arranged between the topsheet and the backsheet, and a joining portion that is provided in a first end portion of body as one end portion of the main-body portion in the waistline direction and joins the first end portion of body to a second end portion of body as the other end portion of the main-body portion in the waistline direction. The main-body portion has an indicator which is arranged between the backsheet and the absorbent core and changes color when coming into contact with moisture. The indicator extends in the waistline direction. In the waistline direction, a length of the indicator is 50% or more of a length of a product region that extends between an end edge of the main-body portion on the side of the second end portion of body and the joining portion. In a diaper to be wrapped around the waist of a pet, an indicator is arranged along the waistline direction. Body fluid is drained to the abdomen. The indicator reacts in the abdomen. When the pet walks on four legs, it is difficult for the user (owner) to see the abdomen, and sometimes the user cannot check the color reaction of the indicator. The length of the aforementioned indicator is 50% or more of the length of the product region, and in a state where the pet diaper is loosely wrapped around the pet's waist, the indicator is arranged in a region that takes up 50% or more of the diaper in the waistline direction. Therefore, in a state where the pet is in the diaper, the indicator is arranged not only in the pet's abdomen but also in the side portion of the pet. Therefore, the user can easily see the indicator when the pet is in the diaper.

According to a preferable aspect, the main-body portion has four equally divided regions determined by dividing the product region in quarters in the waistline direction. The four equally divided regions have a first product region closest to a side of the first end portion of body, and a second product region closest to the side of the second end portion of body. The indicator is arranged in at least one of the first product region and the second product region. When a pet is in the diaper, the first product region and the second product region tend to be arranged on the side portion or the back portion of the pet. In a case where the indicator is arranged in such a region, the visibility of the indicator can be further improved when the pet is in the diaper. Furthermore, the indicator extends to the end portion side of the main-body portion in the waistline direction. Therefore, it is easy for the user to see how far urine has spread in the waistline direction, which makes it possible to inhibit the leakage of body fluid.

According to a preferable aspect, the indicator is arranged in both the first product region and the second product region. In a case where a dog urinates with raising his/her leg, due to the effect of gravity, urine tends to spread to the first product region or the second product region. Because different dogs like to raise different legs, arranging the indicator on both the regions makes it possible to prevent leakage.

According to a preferable aspect, in the waistline direction, a length of the indicator is 50% or more of a length of the absorbent core. In a diaper wrapped along the waistline direction of a pet, the region where the absorbent core is arranged is not easily seen when the pet is in the diaper. If the body fluid spreads to a portion without the absorbent core, the fur on the back is likely to wet. In a case where the user can see that urine has spread over 50% or more of the area of the absorbent core, the user can find out the timing of replacement, which makes it possible to prevent fur wetting or leakage.

According to a preferable aspect, in the waistline direction, a length of the indicator is 80% or more of a length of the absorbent core. According to the present aspect, the user can more appropriately notice that body fluid has spread to the end edge of the absorbent core in the waistline direction.

A pet diaper according to another aspect has a waistline direction arranged along a waistline direction of a pet and a cross direction which is orthogonal to the waistline direction and extends in a direction connecting a belly side and a back side of a pet, the pet diaper comprising a main-body portion that has a topsheet, a backsheet, and an absorbent core arranged between the topsheet and the backsheet, and a tail hole through which the pet's tail can pass. The main-body portion has an indicator which is arranged between the backsheet and the absorbent core and changes color when coming into contact with moisture. The indicator extends in the cross direction. The pet diaper has a space region as a space between the absorbent core and the tail hole apart from each other in the cross direction. The indicator is arranged straddling a core region that overlaps the absorbent core and the space region. If body fluid reaches the tail hole, there is a risk that the body fluid will leak through the tail hole. However, because the indicator is arranged straddling the core region and the space region, the user can notice that the body fluid has spread beyond the absorbent core, which makes it possible to inhibit the leakage of the body fluid.

According to a preferable aspect, in the cross direction, the indicator is arranged in an area taking up 50% or more of the space region. According to the present aspect, by the indicator, the user can more appropriately ascertain how far body fluid has spread within the space region.

According to a preferable aspect, the indicator is arranged straddling a hole region that overlaps the tail hole and the space region. According to the present aspect, the user can notice that body fluid has reached the region that overlaps the tail hole, which makes it possible to inhibit the body fluid from leaking through the tail hole.

According to a preferable aspect, the backsheet has a printing region on which printing is performed and a non-printing region on which nothing is printed. Fifty percent or more of the area of the indicator is arranged in a region that overlaps the non-printing region. According to the present aspect, 50% or more of the area of the indicator overlaps the non-printing region and is less likely to be affected by the printing of the backsheet. Therefore, the visibility of the indicator can be improved.

According to a preferable aspect, the indicator is arranged straddling boundaries between the non-printing region and the printing region that are adjacent to each other. The visibility of the indicator varies between the printing region and the non-printing region. In a case where the way the indicator is seen is varied between portions, it is easy to ascertain the mode of color reaction of the indicator, that is, to what portion the body fluid has spread.

In a manufacturing method of a pet diaper according to one aspect comprises a sheet transport step of transporting a continuous sheet member composed of a continuous sheet member, a mark forming step of forming a marked portion in the continuous sheet member, and a core laminating step of laminating an absorbent core on the continuous sheet member having the marked portion, wherein in the core laminating step, a position of the marked portion on the continuous sheet member is detected, and where the absorbent core is to be laminated is adjusted based on the position of the marked portion. According to the present aspect, the relative positions of the marked portion and the absorbent core can be kept constant. Therefore, the present aspect can inhibit the occurrence of an incident where the absorbent cores in the diapers are put on different positions when the diapers are put on a pet based on the marked portion. In a case where the positional variation of the marked portion in all diapers is inhibited, the diapers can be more accurately positioned by the marked portion.

### (2) Configuration of Pet Diaper

Hereinafter, pet diapers according to embodiments will be described with reference to drawings. It should be noted that, in the following description of the drawings, identical or similar portions will be given identical or similar reference signs. However, the drawings are schematic, and it is noted that the ratios of the respective dimensions and the like are different from the actual ones. Therefore, specific dimensions and the like need to be determined with reference to the following description. In addition, there may be a portion where the relationship or ratio between dimensions does not match between drawings.

In the present specification, “pets” broadly include vertebrates and invertebrates and include typical pets such as cats, dogs, rabbits, and hamsters. The pet diaper of the present embodiment is a dog diaper. In a modified example, the pet diaper may be a cat diaper.

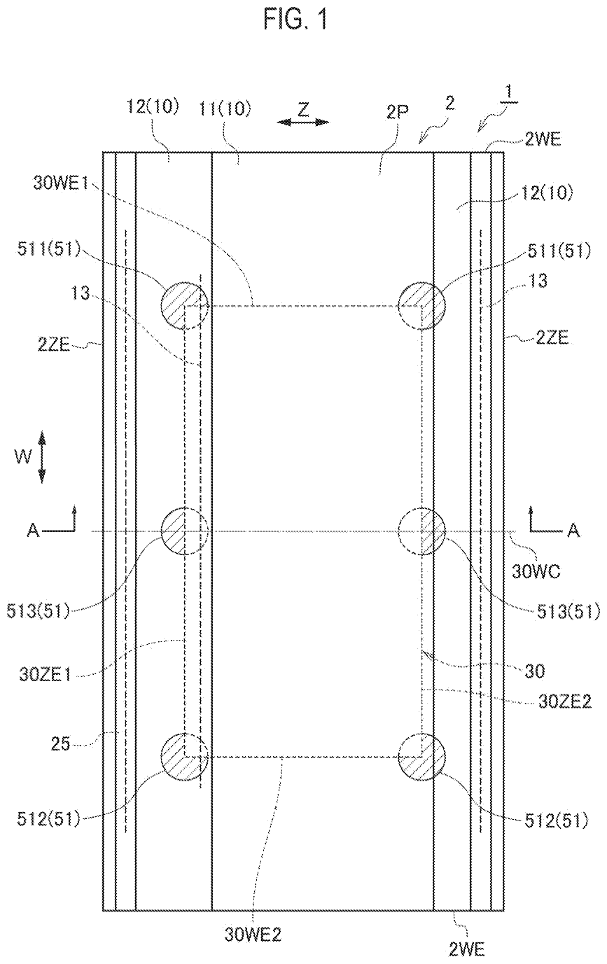

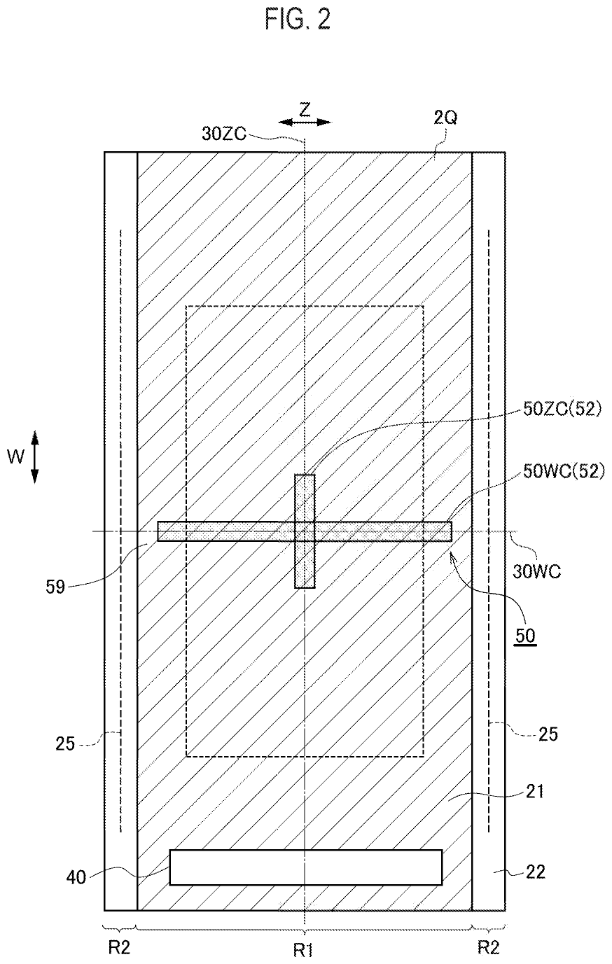

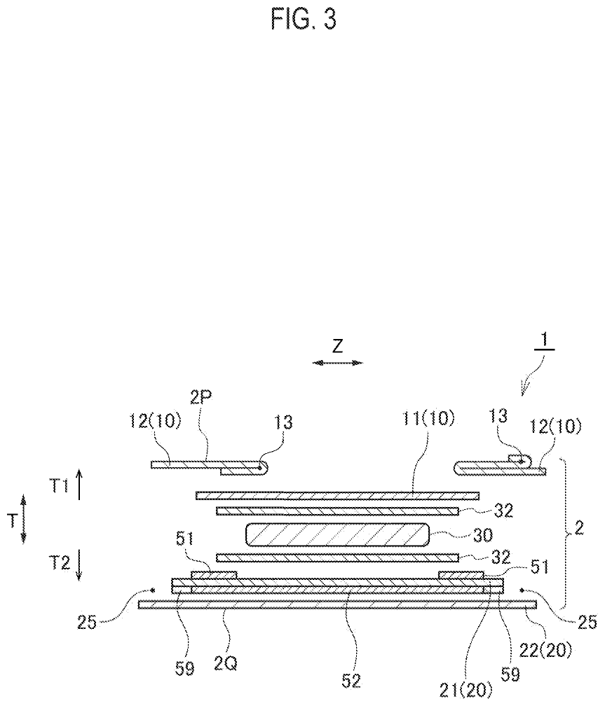

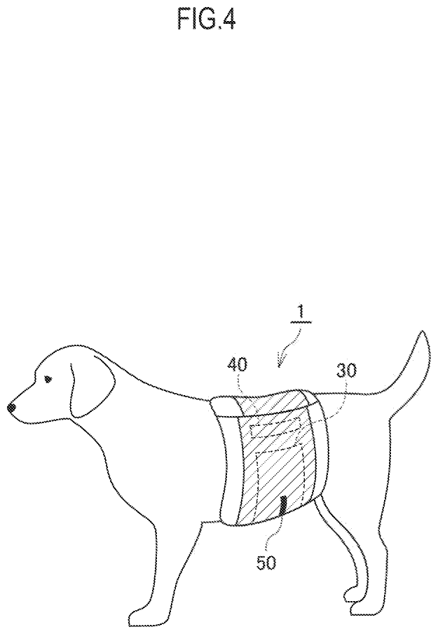

FIG. 1 is a plan view of a pet diaper according to a first embodiment, seen from a skin facing side. FIG. 2 is a plan view of the pet diaper according to the first embodiment, seen from a non-skin facing side. FIG. 3 is a schematic cross-sectional view of the pet diaper taken along the line A-A shown in FIG. 1. FIG. 1 and FIG. 2 show a pet diaper 1 in a stretched state where the diaper is stretched until no wrinkles are formed. In the cross-sectional view shown in FIG. 3, for the convenience of description, each member is illustrated as being spaced apart from each other in a thickness direction T. However, in an actual product, the members are in contact with each other the thickness direction T. FIG. 4 is a view schematically showing the pet diaper worn by a pet.

The pet diaper has a waistline direction W arranged along the waistline direction of a pet, a cross direction Z orthogonal to the waistline direction W, and a thickness direction T orthogonal to the waistline direction W and the cross direction Z. The cross direction Z extends in the longitudinal direction of the pet, that is, in a direction that connects a crotch side and a side away from the crotch. The thickness direction T extends to a top surface side T1 that comes into contact with a pet when the pet is in the diaper and a back surface side T2 that is exposed to the outside when the pet is in the diaper. As shown in FIG. 4, the pet diaper according to the first embodiment is not arranged to cover a region of pet's body that begins at the belly side, passes through the crotch, and reaches the back side. The pet diaper is worn by being wrapped around the pet's waist. Therefore, the length of the pet diaper 1 in the waistline direction W is longer than the length of the pet diaper 1 in the cross direction Z.

The pet diaper 1 has a main-body portion 2 and a joining portion 40. The main-body portion 2 has at least a topsheet 10, a backsheet 20, and an absorbent core 30. The topsheet 10 is arranged on a surface that is brought into contact with a pet. The topsheet 10 has liquid permeability that allows a liquid to pass through toward the absorbent core 30. The topsheet 10 may have a center sheet 11 that is positioned at the center in the cross direction Z and covers the absorbent core 30, and side sheets 12 that cover two side portions of the center sheet 11 in the cross direction Z. The topsheet 10 may be provided with a printing portion. The printing portion may configure a marked portion 50 which will be described later.

As shown in FIG. 3, the side sheets 12 may be folded. Specifically, the side sheet 12 on one end portion side (left side in FIG. 1) in the cross direction Z is folded back toward an outer surface side T2 at the inner edge of the side sheet 12. Furthermore, the side sheet 12 on the other end portion side (right side in FIG. 1) in the cross direction Z is folded back toward a top surface side T1 at the inner edge of the side sheet 12, and the portion folded back is folded back again toward the top surface side T1. A side stretchable member 13 stretched in the waistline direction W may be arranged between the side sheets 12 folded back. The side stretchable member 13 may configure a leak-proof gather that rises up toward the pet side. When the pet is in the diaper, one end portion side in the cross direction Z may be arranged on the front side of the pet, and the other end portion side in the cross direction Z may be arranged on the rear side of the pet. In the present embodiment, the side sheet on the side of the one end portion in the cross direction Z and the side sheet on the side of the other end portion in the cross direction are asymmetric. However, the side sheets may be symmetric in a modified example. Furthermore, in the present invention, “end portion” means a portion that covers a certain region including the end edge.

The absorbent core 30 is arranged between the topsheet 10 and the backsheet 20. The absorbent core 30 is a laminate of absorbent materials such as pulp. A core wrap 32 that covers the absorbent core 30 may be provided. The absorbent core 30 is arranged away from two end edges 2WE of the main-body portion 2 toward the inner side in the waistline direction W. That is, the length of the absorbent core 30 in the waistline direction W is smaller than the length of the main-body portion 2 in the waistline direction W. The absorbent core 30 is arranged at the center of the main-body portion 2 in the waistline direction W, and is not provided in the outer side portion of the main-body portion 2 in the waistline direction W. Furthermore, the absorbent core 30 may be arranged away from two end edges 2ZE of the main-body portion 2 toward the inner side in the cross direction Z. That is, the length of the absorbent core 30 in the cross direction Z may be smaller than the length of the main-body portion 2 in the cross direction Z. The absorbent core 30 may be arranged at the center of the main-body portion 2 in the cross direction Z. and may not be provided in the outer side portion of the main-body portion 2 in the cross direction Z. Furthermore, the absorbent core and the core wrap may be provided with a printing portion. The printing portion may configure the marked portion 50.

The backsheet 20 may have a liquid-impermeable back film 21 and a back-surface nonwoven fabric 22 positioned closer to the outer surface than the back film 21. In a modified example, the backsheet may have a liquid-impermeable back film and a back-surface nonwoven fabric positioned closer to the back surface than the back film 21. The surface of the back film 21 on the top surface side T1 may be provided with an indicator that has a color reaction when coming into contact with body fluid and a printing portion, and the surface of the back film 21 on the outer surface side T2 may be provided with a printing portion. At least one of the indicator and the printing portion may configure the marked portion 50. The length of the back film 21 in the cross direction Z may be smaller than the length of the back-surface nonwoven fabric 22 in the cross direction Z. That is, the back-surface nonwoven fabric 22 may further extend than the back film 21 toward the two sides in the cross direction Z. In a case where the printing portion is provided in the entire back film 21, the region where the back film 21 is arranged configure a printing region R1, and a non-printing region R2 on which nothing is printed may be provided on two sides of the back film 21 in the cross direction Z.

A waist gather that stretches and contracts in the waistline direction W may be arranged at two end portions of the main-body portion 2 in the cross direction Z. The waist gather may be at a position away from the absorbent core 30 toward the outer side in the cross direction Z, and configured with the topsheet 10, the backsheet 20, and a waist stretchable member 25. The waist stretchable member 25 may be arranged between the topsheet 10 and the backsheet 20, and fixed to the topsheet 10 and the backsheet 20 in a state of being stretched in the waistline direction W. The waist stretchable member 25 and the aforementioned side stretchable member 13 configure a stretchable member of the present invention.

When a pet is in the diaper, the joining portion 40 joins the main-body portions 2 that face each other. Specifically, the joining portion 40 is a joining unit for fastening an outer surface 2Q which is a surface of the main-body portion 2 on the side of the backsheet to an inner surface 2P which is a surface of the main-body portion 2 on the side of the topsheet 10. The joining portion 40 of the present embodiment is a mechanical fastener arranged on the outer surface of the main-body portion 2. The joining portion 40 is configured to be engageable with the topsheet 10 of the main-body portion. The joining portion 40 is provided at one end portion of the main-body portion 2 in the waistline direction W. When the pet diaper 1 is put on a pet, the diaper is arranged so that the joining portion 40 is covered with the other end portion of the main-body portion 2 in the waistline direction. In this way, the joining portion 40 can be engaged with the topsheet 10, and the pet can keep on wearing the pet diaper. In a modified example, the joining portion may be arranged on the inner surface of the main-body portion and configured to be engageable with the backsheet of the main-body portion.

Next, the marked portion 50 will be described. The marked portion 50 functions as a guide showing where the pet diaper 1 is to be put on. The sizes of pets vary with the types of pets. Furthermore, due to molting, the sizes of pets vary with the seasons. Therefore, sometimes it is difficult for the user to put the diaper on a pet in a way adapted for the pet's body shape. However, according to the present embodiment, the user can position the pet diaper 1 based on the marked portion 50. Therefore, the pet diaper 1 can be put on a proper position. Furthermore, even if the user cannot put the diaper on a proper position or the diaper is displaced after being worn, the user can notice the displacement or twisting of the diaper based on the marked portion 50, which enables the user to properly correct the position or adjust the diaper to an appropriate state.

Specifically, as shown in FIG. 4, the waistline direction W of the main-body portion 2 of the pet diaper 1 is aligned with the waist of a pet, and the pet diaper 1 is worn by being wrapped around the pet's waist. At this time, the absorbent core 30 is not arranged in the entire region of the main-body portion 2 in the waistline direction W. Furthermore, the absorbent core 30 is not arranged in two side portions of the main-body portion 2 in the waistline direction. For example, in a case where the diaper is worn so that the outer side portion of the main-body portion 2 in the waistline direction W (a portion where the absorbent core is not arranged) faces the pet's excretion opening, the absorbent core cannot absorb body fluid, and leakage occurs. However, based on the marked portion 50, the absorbent core 30 can be arranged at an appropriate position where the absorbent core 30 can absorb excrement, which makes it possible to inhibit the leakage of excrement.

The marked portion 50 of the present embodiment may have a first marked portion 51 shown in FIG. 1 and a second marked portion 52 shown in FIG. 2. The first marked portion 51 is configured to be visible from the topsheet side (inner surface side) of the pet diaper. The topsheet side of the pet diaper is the side of a surface brought into contact with a pet when the diaper is put on the pet. The first marked portion 51 makes it possible to position the diaper while bringing the first marked portion 51 into contact with the pet's body.

The first marked portion 51 may be a printing portion provided in the topsheet 10, a printing portion provided in the absorbent core 30 or the core wrap 32, or a printing portion or an indicator which is provided in the backsheet 20 and configured to be visible through a low-basis-weight portion of the absorbent core 30. The low-basis-weight portion is a portion where the basis weight of the absorbent material is lower than the surrounding portion or a portion where the basis weight of the absorbent material is 0.

The second marked portion 52 is configured to be visible from the backsheet side (outer surface side) of the pet diaper 1. When the user puts the pet diaper on a pet, the backsheet side of the diaper is positioned on the outer side. Furthermore, even after the diaper is worn by the pet, the backsheet is visible. Therefore, the user can position the diaper not only in the process of putting the diaper on the pet but also after putting the diaper on the pet. Even though the diaper turns in the waistline direction and is displaced when put on a pet, the user can notice the displacement based on the marked portion and can properly correct the position.

The second marked portion 52 may be a printing portion provided in the backsheet 20 or an indicator provided in the backsheet 20 or the core wrap 32. The whole outer surface of the back film 21 of the present embodiment is provided with a printing portion. The printing portion may configure a marked portion as well as a decorative portion. As shown in FIGS. 2 and 3, in the present embodiment, a surface of the back film on the side of the outer surface is provided with a printing portion that configures the second marked portion 52 and a printing portion that configures a decorative portion 59. The decorative portion is a portion having a design other than the marked portion, and includes not only a design having symbols or patterns but also background color consisting of only color.

In the present invention, “visible” means that a test subject having good eye sight (1.0 or more) for both eyes can visually recognize an object in a case where the test subject sees the object at a distance of about 30 cm to 50 cm in a room (scale: 500 lx to 750 lx (Lux)) illuminated with a bright natural white light (scale of color temperature: 4,600 K to 5,400 K (Kelvin)). The marked portion 50 may have at least any one of the first marked portion 51 and the second marked portion 52.

As shown in FIG. 2, the second marked portion 52 may have a waist center marked portion 50WC that indicates a center 30WC of the absorbent core 30 in the waistline direction W. The center of the absorbent core in the waistline direction W can be aligned with the pet's excretion opening. Therefore, in the pet diaper in which the absorbent core 30 is short in the waistline direction, the absorbent core is arranged at a position where the absorbent core can absorb excrement, which makes it possible to inhibit the leakage of excrement. The waist center marked portion 50WC of the present embodiment passes through the center 30WC of the absorbent core 30 in the waistline direction W and extends across the entire region of the absorbent core 30 in the cross direction Z. Therefore, the center 30WC of the absorbent core 30 in the waistline direction W is visible within the entire region of the absorbent core 30 in the cross direction Z, which makes it easier for the user to ascertain the central position of the absorbent core 30. The waist center marked portion 50WC may be arranged on the center 30WC of the absorbent core 30 in the waistline direction W, and may be a point or a pattern provided at the center 30WC of the absorbent core in the waistline direction W.

Furthermore, the second marked portion 52 may have a cross center marked portion 50ZC that indicates a center 30ZC of the absorbent core 30 in the cross direction Z. In the pet diaper in which the absorbent core 30 is short in the cross direction Z, the absorbent core 30 can be arranged at a position where the absorbent core 30 can absorb excrement, which makes it possible to inhibit the leakage of excrement. The cross center marked portion 50ZC may be in the form of a line that passes through the center 30ZC of the absorbent core 30 in the cross direction Z and extends in the waistline direction W, or may be a point or pattern arranged at the center 30ZC of the absorbent core 30 in the cross direction Z. In a region where the waist center marked portion 50WC and the cross center marked portion 50ZC are arranged overlapping the absorbent core 30, the portions 50WC and 50ZC may be configured with an indicator showing the absorption of body fluid into the absorbent core.

As shown in FIG. 1, a plurality of first marked portions 51 may be arranged in the waistline direction W by being spaced apart from each other. Because the plurality of first marked portions 51 is arranged along the waistline direction W, the user can position the diaper based on the first marked portions 51 in various directions (for example, by seeing the marked portions from the right side or the left side). Furthermore, the user can notice the displacement occurring in various directions, such as the displacement or twisting on the right side and the displacement or twisting on the left side, and can adjust the diaper to an appropriate state. In a case where the first marked portions 51 are spaced apart in the waistline direction W, based on the upper surface (apex of the back), the lower surface (apex of the bulging belly), the left lateral surface (space between the upper surface and the lower surface on the left side in a front view), and the right lateral surface (space between the upper surface and the lower surface on the right side in a front view) of a pet who walks on four legs and is seen from the front, the user can align the diaper with the positions in the pet's body.

The first marked portion 51 may have a central marked portion 513, a first waist side marked portion 511, and a second waist side marked portion 512. The central marked portion 513 and the waist side marked portions 511 and 512 may be arranged in the waistline direction W by being spaced apart from each other. The central marked portion 513 configures the waist center marked portion 50WC positioned at the center 30WC of the absorbent core 30 in the waistline direction W. The first waist side marked portion 511 and the second waist side marked portion 512 are arranged in the waistline direction W on two sides facing each other across the center of the absorbent core 30, and configure a pair of waist side marked portions of the present invention. The pair of waist side marked portions are arranged in the waistline direction W on two sides facing each other across the center 30WC of the absorbent core 30, and can indicate the position of the absorbent core in the waistline direction W. Therefore, the absorbent core 30 can be appropriately arranged in the waistline direction W, which makes it possible to inhibit the leakage of excrement.

As shown in FIG. 1, one of the pair of waist side marked portions, the first waist side marked portion 511, may be arranged in a region that overlaps one end portion of the absorbent core 30 in the waistline direction W, and the other, the second waist side marked portion 512, may be arranged in a region that overlaps the other end portion of the absorbent core 30 in the waistline direction W. Two end portions of the absorbent core 30 in the waistline direction W are portions to which body fluid reaches immediately before leaking. The user can ascertain the positions of the two end portions of the absorbent core 30 in the waistline direction W, and can arrange the absorbent core 30 so that leakage does not occur.

The first waist side marked portion 511 may be arranged away from one end edge 30WE1 of the absorbent core 30 in the waistline direction W toward the outer side in the waistline direction W. The second waist side marked portion 512 may be arranged away from the other end edge 30WE2 of the absorbent core 30 in the waistline direction W toward the outer side in the waistline direction W. By the marked portion 50 arranged on the outer side in the waistline direction W, the user can ascertain the position of the region where the absorbent core 30 is not arranged, and can arrange the absorbent core 30 so that leakage does not occur.

The marked portion 50 that is arranged away from one end edge 30WE1 of the absorbent core 30 in the waistline direction W toward the outer side in the waistline direction W and the marked portion 50 that overlaps one end portion of the absorbent core 30 in the waistline direction W may be integrated or may be separated from each other. Likewise, the marked portion 50 that is arranged away from the other end edge 30WE2 of the absorbent core 30 in the waistline direction W toward the outer side in the waistline direction W and the marked portion 50 that overlaps the other end portion of the absorbent core 30 in the waistline direction W may be integrated or may be separated from each other.

It is preferable that each of the first waist side marked portion 51 and the second waist side marked portion 512 straddle the end edges of the absorbent core 30 in the waistline direction W or straddle the end edges of the absorbent core 30 in the cross direction Z. The first marked portion 51 of the present embodiment is configured with a printing portion provided on the inner surface of the back film 21. The first marked portion SI is arranged in a region that overlaps the absorbent core 30 and a region that does not overlap the absorbent core 30. When the first marked portion 51 is seen from the inner surface side of the pet diaper, the first marked portion 51 in a region that does not overlap the absorbent core 30 is highly visible while the first marked portion 51 in a region that overlaps the absorbent core 30 is less visible. Therefore, a part of the first marked portion SI stands out. The difference in visibility of the first marked portion 51 makes it easier to ascertain the position of the end edge of the absorbent core.

A plurality of first marked portions 51 may be arranged in the cross direction Z by being spaced apart from each other. Specifically, the central marked portions 513 may be arranged in the cross direction Z by being spaced apart from each other, the first waist side marked portions 511 may be arranged in the cross direction Z by being spaced apart from each other, and the second waist side marked portions 512 may be arranged in the cross direction Z by being spaced apart from each other. Because the plurality of marked portions is arranged along the cross direction Z, the user can position the diaper based on the marked portions in various directions (for example, by seeing the marked portions from the front side or the rear side).

The first marked portion SI may be arranged on two sides facing each other across the center 30WC of the absorbent core 30 in the cross direction Z. Specifically, a pair of central marked portions 513 may be arranged on two sides of the center 30WC of the absorbent core 30, a pair of first waist side marked portions 511 may be arranged on two sides of the center 30WC of the absorbent core 30, and a pair of second waist side marked portions 512 may be arranged on two sides of the center 30WC of the absorbent core 30. Therefore, the pair of central marked portions 513, the pair of first waist side marked portions 511, and the pair of second waist side marked portions 512 function as “a pair of cross side marked portions” of the present invention, respectively. By the cross side marked portions arranged on two sides facing each other across the center of the absorbent core 30 in the cross direction Z, the user can easily ascertain the position of the absorbent core 30 in the cross direction Z. Accordingly, the user can appropriately arrange the absorbent core 30 in the cross direction Z, which makes it possible to inhibit the leakage of excrement.

One of the pair of cross side marked portions may be arranged in a region that overlaps one end portion of the absorbent core 30 in the cross direction Z, and the other may be arranged in a region that overlaps the other end portion of the absorbent core 30 in the cross direction Z. Specifically, as shown in FIG. 1, between the pair of first waist side marked portions 511, the first waist side marked portion 511 on one end portion side (left side in FIG. 1) in the cross direction Z may be arranged in a region that overlaps one end portion of the absorbent core 30 in the cross direction Z, and the first waist side marked portion 511 on the other end portion side (right side in FIG. 1) in the cross direction Z may be arranged in a region that overlaps the other end portion of the absorbent core 30 in the cross direction Z. The user can ascertain the positions of two end portions of the absorbent core 30 in the cross direction Z, and can arrange the absorbent core so that leakage does not occur.

Furthermore, one of the pair of cross side marked portions may be arranged away from one end edge 30ZE1 of the absorbent core 30 in the cross direction Z toward the outer side in the cross direction Z, and the other may be arranged away from the other end edge 30ZE2 of the absorbent core 30 in the cross direction Z toward the outer side in the cross direction Z. Specifically, the first waist side marked portion 511 on one end portion side (left side in FIG. 1) in the cross direction Z may be arranged away from one end edge 30ZE1 of the absorbent core 30 in the cross direction Z toward the outer side in the cross direction Z, and the first waist side marked portion 511 on the other end portion side (right side in FIG. 1) in the cross direction Z may be arranged away from the other end edge 30ZE2 of the absorbent core 30 in the cross direction Z toward the outer side in the cross direction Z. By the marked portion 50 arranged on the outer side in the cross direction Z, the user can ascertain the position of a region where the absorbent core 30 is not arranged, and can arrange the absorbent core 30 so that leakage does not occur.

As shown in FIG. 2, the backsheet 20 may be provided with the printing region R1 on which a design including the marked portion 50 is printed and a non-printing region R2 on which nothing is printed. The non-printing region R2 may be provided in at least at two end portions of the backsheet 20 in the cross direction Z. By the boundary between the non-printing region R2 and the printing region R1, the user can ascertain two end portions of the main-body portion 2 in the cross direction Z. Furthermore, in a case where the non-printing region R2 is provided, the printing region R1 easily stands out, which can further improve the visibility of the marked portion 50 in the printing region R1. The boundary between the non-printing region R2 and the printing region R1 may extend along the waistline direction W and be positioned away from the absorbent core 30 toward the outer side in the cross direction Z. That is, the non-printing region R2 may be arranged only on a side away from the absorbent core 30 toward the outer side in the cross direction Z. By the non-printing region R2, the user can ascertain the region where the absorbent core 30 is not arranged, and can arrange the absorbent core 30 so that leakage does not occur.

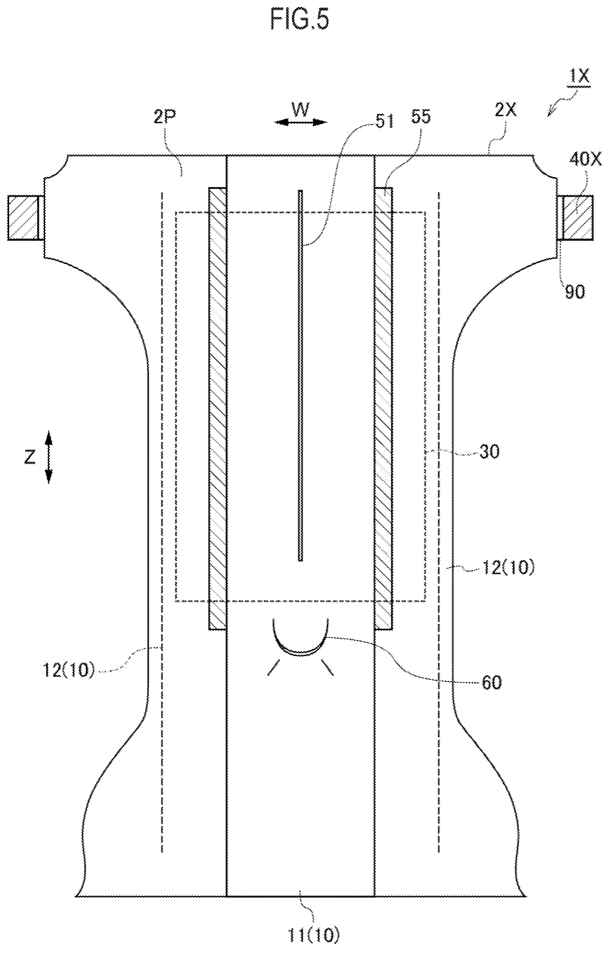

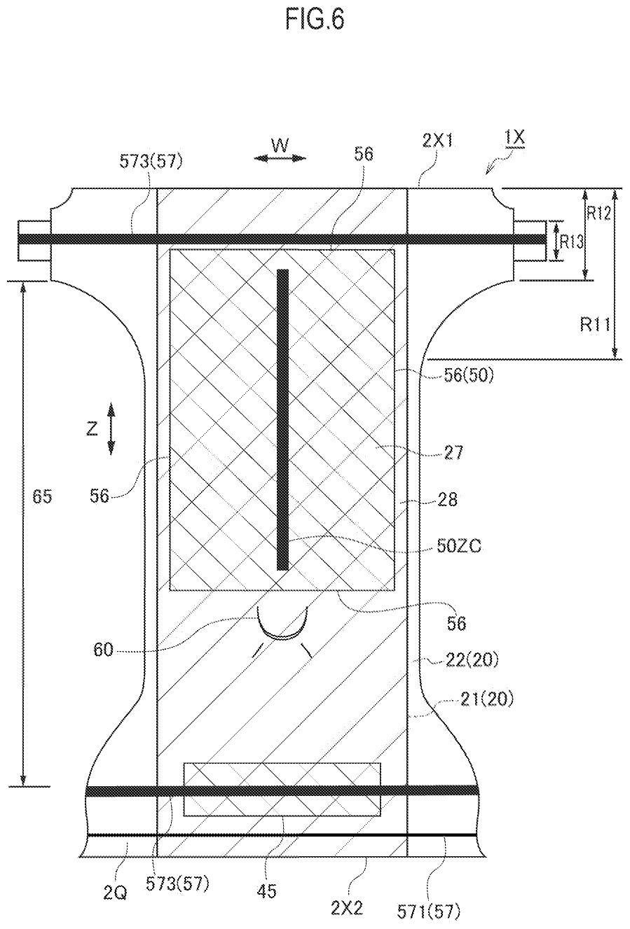

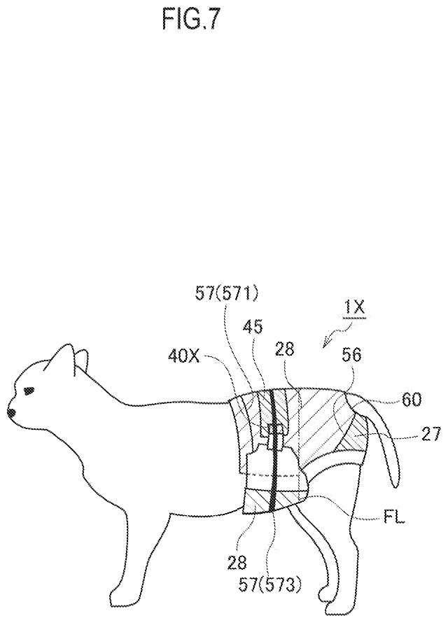

Next, a pet diaper 1X according to a second embodiment will be described. The pet diaper according to the second embodiment is a cat diaper. In a modified example, the pet diaper may be a dog diaper. In the following description of other embodiments, configurations similar to those of the first embodiment will be denoted by the same reference numerals, and description thereof will not be repeated. FIG. 5 is a plan view of the pet diaper according to the second embodiment, seen from a skin facing side. FIG. 6 is a plan view of the pet diaper 1X according to the second embodiment, seen from a non-skin facing side. FIG. 7 is a view schematically showing the pet diaper 1X according to the second embodiment that is worn by a pet.

The pet diaper 1X has a main-body portion 2X and a joining portion 40X. The pet diaper 1X of the second embodiment is worn so as to cover a region of pet's body that begins at the belly side, passes through the crotch, and reaches the back side. Therefore, in the cross direction Z, the length of the pet diaper 1X of the second embodiment is greater than the length of the pet diaper of the first embodiment. The length of the main-body portion 2X in the cross direction Z is greater than the length of the main-body portion 2X in the waistline direction W. The joining portion 40X is provided on a fastening tape 90 that extends from two sides of the main-body portion 2X toward the outer side in the waistline direction W at one end portion of the main-body portion 2X in the cross direction Z. The joining portion 40X is arranged on the inner surface 2P of the main-body portion 2X, and is configured to be joinable to a target portion 45 provided on the outer surface 2Q of the main-body portion 2. In a modified example, the main-body portion may not include the target portion, and the joining portion 40X may be configured to be joined to the backsheet of the main-body portion 2X on the side of the outer surface 2Q. The side of the main-body portion 2X to be brought into contact with the belly side may be provided with the joining portion 40X that extends from 2 sides of the main-body portion 2X toward the outer side in the waistline direction W. Alternatively, the side of the main-body portion 2X to be brought into contact with the back side may be provided with the joining portion 40X that extends from 2 sides of the main-body portion 2X toward the outer side in the waistline direction W.

When the pet diaper 1X is put on a pet, one end portion of the main-body portion 2X (end portion on the side provided with the joining portion 40X) in the cross direction Z is brought into contact with the belly of the pet. At this time, the other end portion of the main-body portion 2X in the cross direction Z is passed between two legs of the pet and exposed to the rear side of the pet. Then, the center of the main-body portion 2X in the cross direction Z is brought into contact with the pet's excretion opening, and the buttocks and back of the pet are covered with the other end portion of the main-body portion 2X in the cross direction Z. Thereafter, the joining portion 40X is pulled toward the back side of the pet and fastened to the outer surface of the target portion 45 at the other end portion of the main-body portion positioned on the back side in the cross direction. In this way, the pet diaper 1X can be worn so as to cover the belly, back, and crotch of the pet as shown in FIG. 7. It is preferable that the pet diaper 1X be arranged so as to cover a region of pet's body that begins at the belly side, passes through the crotch, and reaches the back side, and the cross direction of the main-body portion be appropriately aligned with respect to the center of the pet in the lateral direction. Therefore, the pet diaper 1X may have the cross center marked portion 50ZC that indicates the center 30ZC of the absorbent core 30 in the cross direction Z. The cross center marked portion 50ZC may be configured with an indicator.

The pet diaper 1X may be provided with a tail hole 60 through which the pet's tail can pass. When the diaper having the tail hole 60 is put on a pet, the pet's tail may be passed through the tail hole (A) in the process of covering the buttocks and back of the pet with the other end portion of the main-body portion 2X in the cross direction Z.

As shown in FIG. 5, the marked portion 50 may have a stretching marked portion 55 arranged in a region that overlaps the stretchable member. The stretching marked portion 55 according to the present embodiment is arranged in a region that overlaps the side stretchable member 13. When seen in a plan view, the stretching marked portion 55 may overlap at least a part of the stretchable member. The stretchable member stretches and contracts in at least one of the waistline direction W and the cross direction Z, and is put on a pet so as to be in close contact with the pet's body. Because the stretching marked portion 55 is arranged in the region that overlaps the stretchable member, the user can ascertain the position of the stretchable member that is in close contact with the pet's body, and can arrange the stretchable member at an appropriate position.

As shown in FIG. 6, the marked portion 50 may have a boundary marked portion 56. Specifically, a first decorative portion 27 and a second decorative portion 28 which is different from the first decorative portion 27 in design are printed on the back film 21. The boundary between the First decorative portion 27 and the second decorative portion 28 configure the boundary marked portion 56. The first decorative portion 27 is arranged in a region that overlaps the absorbent core 30, and the second decorative portion 28 is arranged in a region that does not overlap the absorbent core. The boundary marked portion 56 of the present embodiment is provided along the end edge of the absorbent core 30 in the cross direction Z and along the end edge of the absorbent core 30 in the waistline direction W. The present aspect makes it possible to position the diaper based on the marked portion while improving decorativeness by the designs of the two portions, the first decorative portion 27 and the second decorative portion 28.

The pet diaper 1X may have an end edge marked portion 57 that indicates the outer end edge of the main-body portion 2X in the cross direction Z. The end edge marked portion 57 indicates the position of the outer end edge of the main-body portion 2X in the cross direction Z. In a case where the diaper has the end edge marked portion 57, the outer end edge of the main-body portion 2X in the cross direction Z can be appropriately arranged, and the diaper can be worn in a state where the entirety of the main-body portion 2X is appropriately wrapped around the pet's body. Therefore, the diaper can fit better the pet, and discomfort resulting from excessive tightness can be reduced. The end edge marked portion 57 may be a marked portion that functions as a guide helping positioning the outer end edge of the main-body portion 2X in the cross direction Z. Specifically, the end edge marked portion 57 may be a first end edge marked portion 571 showing the position to which one outer end edge 2X1 of the main-body portion 2X in the cross direction Z is to be fitted, a second end edge marked portion (not shown in the drawing) showing the position to which the other outer end edge 2X2 of the main-body portion 2X in the cross direction Z is to be fitted, or a third end edge marked portion 573 that enables the user to tell whether the outer end edge of the main-body portion 2X in the cross direction Z is appropriately arranged. The rust end edge marked portion 571 functions in a case where the diaper is worn so that one outer end edge 2X1 of the main-body portion 2X in the cross direction Z is away from the other outer end edge 2X2 of the main-body portion 2X in the cross direction Z toward the inner side in the cross direction Z. Furthermore, the second end edge marked portion functions in a case where the diaper is worn so that the other outer end edge 2X2 of the main-body portion 2X in the cross direction Z is away from one outer end edge 2X1 of the main-body portion 2X in the cross direction Z toward the inner side in the cross direction Z. It is appropriate that the diaper 1X of the present embodiment is worn so that one outer end edge 2X1 of the main-body portion 2X in the cross direction Z is away from the other outer end edge 2X2 of the main-body portion 2X in the cross direction Z toward the inner side in the cross direction Z. The diaper 1X has the first end edge marked portion 571.

As shown in FIG. 7, the third end edge marked portion 573 is arranged on the one outer end edge 2X1 side of the main-body portion 2X and the other outer end edge 2X2 side of the main-body portion 2X. The third end edge marked portion 573 may be configured so that in a state where the outer end edge of the main-body portion 2X in the cross direction Z is appropriately arranged, the marked portion arranged on the one outer end edge 2X1 side and the marked portion arranged on the other outer end edge 2X2 side coincide in the cross direction Z. In a case where the pet diaper is worn so that outer end edges on two sides of the main-body portion 2X in the cross direction Z coincide, sometimes the diaper does not fit the pet's body shape. In this case, if the end edge marked portion 57 is provided, the user can put the diaper on the pet by misaligning the outer end edges on two sides of the main-body portion 2X in the cross direction Z, and to easily fit the diaper to the pet's body shape. For example, in the case of a diaper for infants to be worn to cover a region of wearer's body that begins at the belly side, passes through the crotch, and reaches the back side, where the diaper is to be put on can be varied in the longitudinal direction. Generally, in this way, the diaper is worn so that one end and the other end in the cross direction coincide. However, in the case of a pet diaper having a tail hole, it is difficult to vary where the diaper is to be put on in the longitudinal direction. Furthermore, in a case where the pet diaper is fitted to the pet's body, sometimes one end and the other end in the cross direction do not coincide. At this time, in a case where the user puts the diaper on the pet so that one end and the other end in the cross direction coincide, unfortunately, the diaper tightens the belly side or hangs down from the belly side. However, according to the present embodiment, based on the positional relationship of the marked portions in the diaper that is properly put on a pet, the user can make the pet to keep on wearing the diaper on an appropriate position.

At least one of the plurality of marked portions 50 may be arranged in a region interposed between a pair of leg opening 65. In a diaper that is to be worn to cover a region of pet's body that begins at the belly side, passes through the crotch, and reaches the back side, it is preferable that the joining portion 40X be fastened along a line (FL in FIG. 7) that extends from the groin area on the belly side toward the back side. In a case where the joining portion 40X is fastened along a line that extends from the groin area on the belly side toward the back side, the diaper fits around the legs, which makes it possible to inhibit leakage or displacement. In a case where the marked portion 50 is provided in the region interposed between the leg opening, the user can fasten the joining portion 40X based on the marked portion 50 in the region around legs.

The end edge marked portion 57 may be arranged in a certain region from the outer end edge of the main-body portion 2X in the cross direction Z. Specifically, the end edge marked portion 57 may be arranged in the first region (R11 shown in FIG. 6) from the outer end edge of the main-body portion 2X in the cross direction Z to the groin area (the boundary between the leg and the buttocks). The inner end edge of the first region R11 in the cross direction Z is a portion to be fitted to the groin area. When positioning the diaper on the groin area, the user can easily recognize the end edge marked portion 57. It is preferable that the end edge marked portion 57 be arranged in a second region (R12 shown in FIG. 6) from the outer end edge of the main-body portion 2X in the cross direction Z to the boundary between the leg and the waist. The inner end edge of the second region R12 in the cross direction Z is a portion to be fitted to the boundary between the waist and the leg. When positioning the diaper around the waist, the user can easily recognize the end edge marked portion 57. The first region R11 and the second region R12 are regions where displacement resulting from leg movements does not easily occur. Once the diaper is worn, displacement is less likely to occur in these regions. The user can correct the displacement of the diaper by using the end edge marked portion 57, which is less likely to change position, as a guide. It is more preferable that the end edge marked portion 57 be arranged in a third region (R13 shown in FIG. 6) where the fastening tape 90 is arranged. The third region R13 is a region that extends between the outer end edges of the fastening tape 90 in the cross direction Z. When fastening the joining portion 40X with holding the fastening tape 90, the user can easily recognize the end edge marked portion 57.

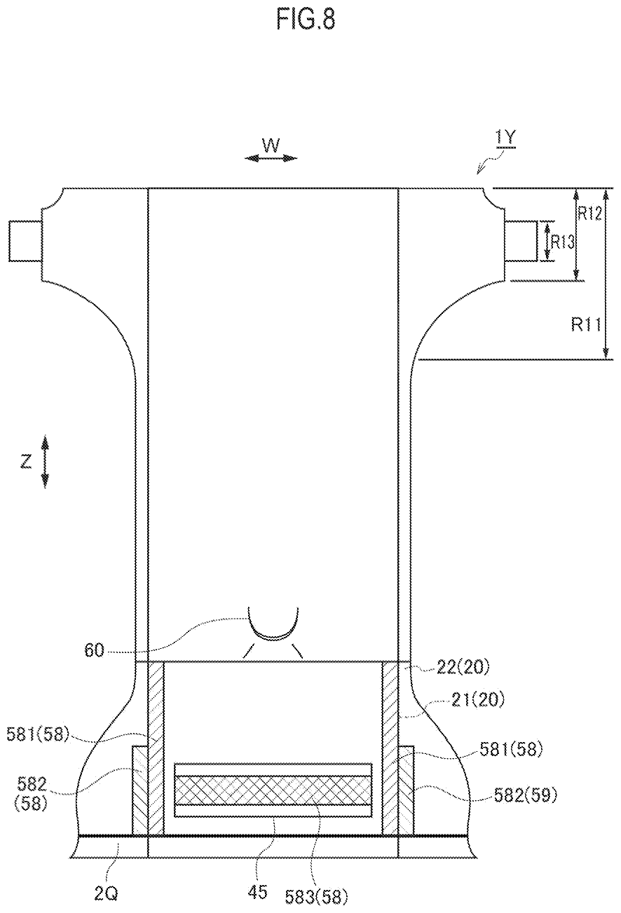

Next, a pet diaper 1Y according to a third embodiment will be described with reference to FIG. 8. FIG. 8 is a plan view of the pet diaper 1Y according to the third embodiment, seen from a non-skin facing side. The configuration of the pet diaper 1Y is the same as that of the diaper 1X of the second embodiment except for marked portions. Therefore, the configuration will be denoted by the same reference numerals, and description thereof will not be repeated. The pet diaper 1Y according to the third embodiment has a region marked portion 58 as a marked portion. The region marked portion is configured to showing where the certain region of the main-body portion 2X extending in the cross direction Z is to be put on.

The region marked portion 58 may have a first region marked portion 581 configured to show where the first region R11, which ranges from the outer end edge of the main-body portion 2X in the cross direction Z to the groin area (boundary between the leg and the buttocks), is to be put on. The first region marked portion 581 may be provided on one side of the main-body portion 2X in the cross direction Z. and show where the first region R11 located on the other side of the main-body portion 2X in the cross direction Z is to be positioned when the diaper is worn. Alternatively, the first region marked portion 581 may be provided on the other side of the main-body portion 2X in the cross direction Z, and show where the first region R11 located on one side of the main-body portion 2X in the cross direction Z is to be positioned when the diaper is worn. The first region marked portion 581 makes it possible to position one end side and the side of the other end of the main-body portion 2X in the cross direction Z. In addition, based on the first region marked portion 581, the diaper can be positioned in the pet's groin area.

The region marked portion 58 may have a second region marked portion 582 showing where the second region R12, which ranges from the outer end edge of the main-body portion 2X in the cross direction Z to the boundary between the leg and the waist, is to be put on. The second region marked portion 582 may be provided on one side of the main-body portion 2X in the cross direction Z. and show where the second region R12 located on the other side of the main-body portion 2X in the cross direction Z is to be positioned when the diaper is worn. Alternatively, the second region marked portion 582 may be provided on the other side of the main-body portion 2X in the cross direction Z, and show where the second region R12 located on one side of the main-body portion 2X in the cross direction Z is to be positioned when the diaper is worn. The second region marked portion 582 makes it possible to position one end side and the side of other end of the main-body portion 2X in the cross direction Z. In addition, based on the second region marked portion 582, the user can position the diaper on the leg and waist of the pet.

The region marked portion 58 may have a third region marked portion 583 showing where the fastening tape 90 is to be put on. The third region marked portion 583 is provided on a side of the main-body portion 2X to which the fastening tape 90 is fastened. The third region marked portion 583 makes it possible to position one end side and the side of the other end of the main-body portion 2X in the cross direction Z. In addition, based on the third region marked portion 583, the relative positions of the target portion 45 and the fastening tape 90 can be appropriately arranged in the cross direction Z.

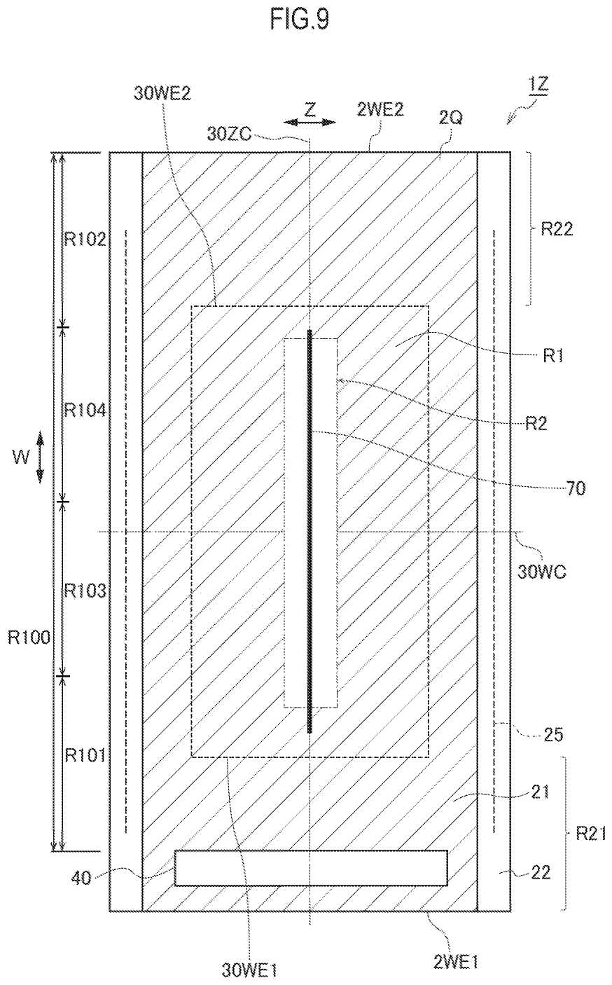

Next, a pet diaper 1Z according to a fourth embodiment will be described. Just as the pet diaper of the first embodiment, the pet diaper 1Z according to the fourth embodiment is worn by being wrapped around the waist. FIG. 9 is a plan view of the pet diaper 1Z according to the fourth embodiment, seen from a non-skin facing side. The main-body portion 2 has a first end portion R21 of body that is one end portion of the main-body portion 2 in the waistline direction W, and a second end portion R22 of body that is the other end portion of the main-body portion 2 in the waistline direction W. The first end portion R21 of body is a region that extends from one end edge 2WE1 of the main-body portion 2 in the waistline direction W toward the inner side in the waistline direction W, and may be a region between one end edge 2WE1 of the main-body portion 2 and one end edge 30WE1 of the absorbent core 30. The second end portion R22 of body is a region that extends from the other end edge 2WE2 of the main-body portion 2 in the waistline direction W toward the inner side in the waistline direction W, and may be a region between the other end edge 2WE2 of the main-body portion 2 and the other end edge 30WE2 of the absorbent core 30. The first end portion R21 of body is provided with the joining portion 40. The joining portion 40 is configured to be engageable with the second end portion R22 of body, and joins the first end portion R21 of body and the second end portion R22 of body.

The main-body portion 2 has an indicator 70 which is arranged between the backsheet 20 and the absorbent core 30 and changes color when coming into contact with moisture. The indicator 70 may be arranged between the back film 21 as the backsheet 20 and the absorbent core 30. The indicator 70 extends in the waistline direction W. The indicator 70 is arranged across the center of the absorbent core 30 in the waistline direction W. When the diaper is put on a pet, the indicator 70 is arranged in at least a portion that comes into contact with the pet's belly. As shown in FIG. 9, the indicator 70 may be in the form of a straight line that extends in the waistline direction W, or may be an assembly of indicator constituent portions arranged in the waistline direction W by being spaced apart from each other. The indicator 70 is in the form of a straight line that extends in the waistline direction W. The diaper may have one indicator 70 as in the present embodiment, or may have a plurality of indicators 70 arranged in the cross direction Z by being spaced apart from each other.

In the waistline direction W, the length of the indicator 70 is 50% or more of the length of a product region R100 that extends between the other end edge 2WE2 of the main-body portion 2 on the side of the second end portion of body and the joining portion 40. The product region R100 is a region that extends in the waistline direction W, between the other end edge 2WE2 of the main-body portion 2 on the side of the second end portion of body and the inner end edge of the joining portion 40 in the waistline direction W. The pet diaper 1Z of the fourth embodiment is configured to be wrapped around the pet's waist, and the indicator 70 is arranged along the waistline direction W. Body fluid is drained to the abdomen where the indicator 70 reacts. However, in a state where the pet walks on four legs, it is difficult for the user (owner) to see the abdomen, and sometimes the user cannot check the color reaction of the indicator 70. The indicator 70 occupies 50% or more of the product region R100, and in a state where the pet diaper 1Z is loosely wrapped around the pet's waist, the indicator 70 is arranged in an area that takes up 50% or more in the waistline direction. Therefore, when the pet is in the diaper, the indicator 70 is arranged not only in the pet's abdomen but also in the pet's side portion.

Accordingly, the user can easily see the indicator 70 when the pet is in the diaper. The main-body portion 2 has four equally divided regions determined by dividing the product region R100 in quarters in the waistline direction W. The four equally divided regions have a first product region R101 closest to the side of the first end portion of body, a second product region R102 closest to the side of the second end portion of body, a third product region R103 adjacent to the first product region R101, and a fourth product region R104 adjacent to the second product region R102. The indicator 70 may be arranged in at least one of the first product region R101 and the second product region R102. When the diaper is put on a pet, the first product region R101 and the second product region R102 tend to be arranged in the side portion or back portion of the pet. In a case where the indicator 70 is arranged in such a region, the visibility of the indicator 70 in the diaper worn by the pet can be further improved. Furthermore, because the indicator 70 extends to the end portion side of the main-body portion 2 in the waistline direction W, the user can easily see how far urine has spread in the waistline direction W, which makes it possible to and inhibit the leakage of body fluid.

The indicator 70 may be arranged in both the first product region R101 and the second product region R102. In a case where a pet urinates with raising his/her leg, due to the effect of gravity, urine tends to spread to the first product region R101 or the second product region R102. Because different dogs like to raise different legs, arranging the indicator on both the regions makes it possible to prevent leakage. The indicator 70 may be arranged in both the third product region R103 and the fourth product region R104. This because the third product region R103 and the fourth product region R104 are arranged in the pet's abdomen and make it easy for body fluid to be directed to these regions. The indicator 70 may straddle the third product region R103 and the first product region R101 or may straddle the fourth product region R104 and the second product region R102. In a case where the indicator 70 straddling a plurality of regions is arranged as above, the user can more easily ascertain how far the body fluid has speared by using the indicator 70.

In the waistline direction W, the length of the indicator 70 may be 50% or more of the length of the absorbent core 30. The indicator 70 is arranged in a region that overlaps the absorbent core 30. It is preferable that in a region overlapping the absorbent core 30 in the waistline direction W, the length of the indicator 70 may be 50% or more of the length of the absorbent core 30. In the case of a diaper that is wrapped along the waistline direction W of a pet, when the pet is in the diaper, it is difficult to see the region where the absorbent core 30 is arranged. If body fluid spreads to a portion without the absorbent core 30, the fur on the back is likely to wet as well. In a case where the user can see that urine has spread over 50% or more of the area of the absorbent core, the user can find out the timing of replacement, which makes it possible to prevent fur wetting or leakage. The length of the indicator 70 is preferably 80% or more of the length of the absorbent core 30 in the waistline direction W, and more preferably 80% or more of the length of the absorbent core 30 in the waistline direction W within a region that overlaps the absorbent core 30. In a case where the diaper has this configuration, the user can ascertain that body fluid has spread to the end edge of the absorbent core 30 in the waistline direction W.

The backsheet 20 has a printing region R1 on which printing is performed and a non-printing region R2 on which nothing is printed. The printing region R1 is a region where printing is performed on at least any one of the back film 21 and the back-surface nonwoven fabric 22 in a plan view. The non-printing region R2 is a region where nothing is printed on both the back film 21 and the back-surface nonwoven fabric 22 in a plan view. At least a part of the indicator 70 is arranged in a region that overlaps the non-printing region R2. In a case where the entirety of the indicator 70 overlaps the printing region R1, there is a risk that it will be difficult to secure the visibility of the indicator 70 due to printing. However, at least a part of the indicator 70 is not affected by the printing of the backsheet 20, which makes it easier to secure the visibility of the indicator 70. It is preferable that 50% or more of the area of the indicator 70 be arranged in a region that overlaps the non-printing region R2. Fifty percent or more of the area of the indicator 70 is not easily affected by the printing of the backsheet 20. Accordingly, the visibility of the indicator 70 can be improved.

The indicator 70 may be arranged straddling the boundaries between the non-printing region R2 and the printing region R1 that are adjacent to each other. The visibility of the indicator 70 varies between the printing region R1 and the non-printing region R2. In a case where the way the indicator 70 is seen is varied between portions, it is easy to ascertain the mode of color reaction of the indicator 70, that is, to what portion the body fluid has spread. The non-printing region R2 of the present embodiment is surrounded by the printing region R1 on all sides. In a plan view, the boundaries between the non-printing region R2 and the printing region R1 that are adjacent to each other form a rectangle long in the waistline direction. The indicator 70 is arranged straddling the boundaries positioned at two ends of the non-printing region R2 in the waistline direction W, among the boundaries between the non-printing region R2 and the printing region R1 that are adjacent to each other.

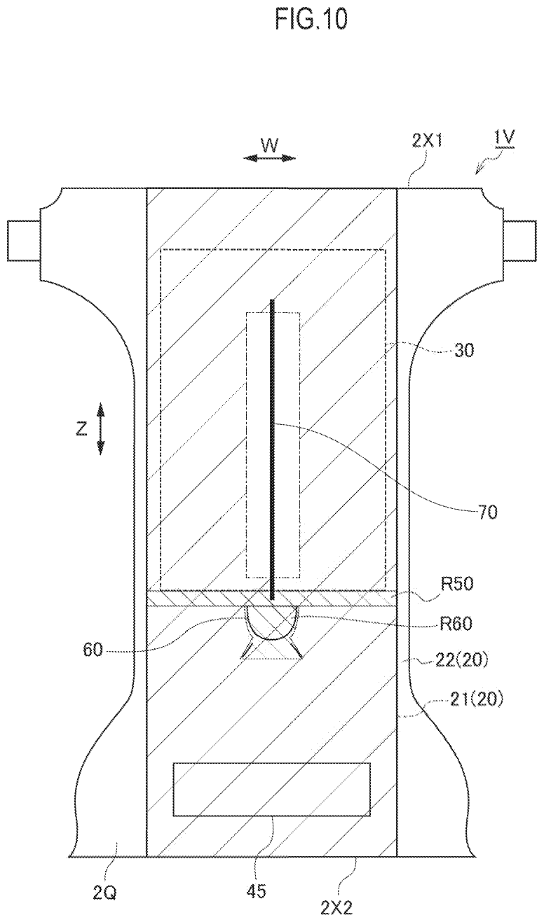

Next, a pet diaper 1V according to a fifth embodiment will be described. Just as the pet diaper of the second embodiment, the pet diaper 1V according to the fifth embodiment is worn to cover a region of pet's body that begins at the belly side, passes through the crotch, and reaches the back side. FIG. 10 is a plan view of the pet diaper 1V according to the fifth embodiment, seen from a non-skin facing side. The pet diaper 1V has a tail hole 60 through which the pet's tail can pass. The main-body portion 2 has an indicator 70 which is arranged between the backsheet 20 and the absorbent core 30 and changes color when coming into contact with moisture. The indicator 70 may be arranged between the back film 21 as the backsheet 20 and the absorbent core 30. The indicator 70 extends in the cross direction Z. The indicator 70 is arranged across the center of the absorbent core 30 in the cross direction Z. When the diaper is put on a pet, the indicator 70 is arranged in at least a portion that comes into contact with the pet's belly. As shown in FIG. 10, the indicator 70 may be in the form of a straight line that extends in the cross direction Z, or may be an assembly of indicator constituent portions arranged in the cross direction Z by being spaced apart from each other. The indicator 70 of the present embodiment is in the form of a straight line that extends in the cross direction Z. The diaper may have one indicator 70 as in the present embodiment, or may have a plurality of indicators 70 arranged in the waistline direction W by being spaced apart from each other.

The pet diaper 1V has a space region R50 as a space between the absorbent core 30 and the tail hole 60 that are apart from each other in the cross direction Z. The indicator 70 is arranged straddling the core region R30 that overlaps the absorbent core 30 and the space region. The core region is a region where the absorbent core 30 is arranged in a plan view. If body fluid reaches the tail hole 60, there is a risk that the body fluid will leak through the tail hole. However, because the indicator 70 is arranged straddling the core region and the space region R50, the user can notice that the body fluid has spread beyond the absorbent core 30, which makes it possible to inhibit the leakage of the body fluid. In the present embodiment, the absorbent core 30 is arranged only on the side closer to one outer end edge 2X1 (joining portion side) of the main-body portion 2X in the cross direction Z than the tail hole 60. Therefore, the space region R50 is arranged only on one side of the tail hole 60 in the cross direction Z. More specifically, the space region R50 extends between the end edge of the tail hole 60 on the side of the outer end edge 2X1 and the end edge of the absorbent core 30 on the side of the outer end edge 2X2. However, in a modified example, in a case where the absorbent core 30 is arranged not only on a side closer to the outer end edge 2X1 than the tail hole 60 but also on a side closer to the outer end edge 2X2 than the tail hole 60, the space region R50 is arranged on two sides of the tail hole 60 in the cross direction Z. In a case where the diaper has a plurality of space regions R50, the indicator 70 may be arranged in any of the space regions R50.

The indicator 70 may be arranged in an area that takes up 50% or more of the space region R50 in the cross direction Z. This aspect makes it possible to more appropriately ascertain how far body fluid has spread within the space region R50) by using the indicator 70. The indicator 70 may be arranged straddling a hole region R60 that overlaps the tail hole 60 and the space region R50. The hole region R60 is a region surrounded by the outline of the tail hole 60, and is indicated by a hatched portion in FIG. 10. According to the present aspect, the user can notice that body fluid has reached the region that overlaps the tail hole 60, which makes it possible to further inhibit the leakage of the body fluid through the tail hole 60.

Just as the indicator according to the fourth embodiment, the indicator 70 according to the fifth embodiment is arranged to overlap the non-printing region R2. Fifty percent or more of the area of the indicator 70 may be arranged in a region that overlaps the non-printing region R2. The indicator 70 may be arranged straddling the boundaries between the non-printing region R2 and the printing region R1 that are adjacent to each other.

Next, an example of a manufacturing method of the pet diaper configured as above will be described. The manufacturing method of the pet diaper may have a sheet transport step of transporting a continuous sheet member composed of a continuous sheet member, a mark forming step of forming the marked portion 50 by performing printing on the continuous sheet member, and an absorbent core laminating step of laminating the absorbent core 30 on the continuous sheet member having the marked portion 50.

In the sheet transport step of transporting the continuous sheet member composed of the continuous sheet member, the continuous sheet member composed of the continuous sheet member configuring any of the topsheet 10, the backsheet, and the core wrap is transported. In the mark forming step, the marked portion 50 is formed on the continuous sheet member. The marked portions 50 may be formed by printing or embossing. In the core laminating step, the position of the marked portion 50 on the continuous sheet member is detected. The position of the marked portion 50 may be detected by an imaging unit such as a camera. In the core laminating step, based on the position of the marked portion 50, where the absorbent core 30 is to be laminated is adjusted. To adjust where the absorbent core 30 is to be laminated is to adjust the positions of the absorbent core 30 and the continuous sheet member. Specifically, adjusting the amount of the continuous sheet member fed out makes it possible to adjust the positions of the continuous sheet member and the absorbent core 30 in transport direction. Furthermore, an adjustment mechanism that comes into contact with the side ends of the continuous sheet member in the transverse direction orthogonal to the transport direction may be provided, and the position of the continuous sheet member in the transverse direction may be adjusted by the adjustment mechanism. According to this manufacturing method, the relative positions of the marked portion 50 and the absorbent core 30 can be kept constant. Therefore, the occurrence of an incident can be inhibited where the absorbent core 30 in the diapers is put on different positions when the diapers are put on the pet based on the marked portion 50. In a case where the positional variation of the marked portions 50 in all diapers is inhibited, the diaper can be more accurately positioned based on the marked portion 50.

Next, a container including a package piece accommodating a plurality of pet diapers will be described. The container has a plurality of pet diapers and a package piece accommodating the plurality of pet diapers. The plurality of pet diapers has a first diaper having a backsheet on which a first decorative portion is printed, and a second diaper having a backsheet on which a second decorative portion different from the first decorative portion in design is printed. The two portions, the first decorative portion and the second decorative portion, mentioned herein may have different designs. The designs of these portions may be different from those of the first decorative portion and the second decorative portion of the second embodiment. In the present embodiment, the first decorative portion or the second decorative portion is printed on the back film of the backsheet. According to the present embodiment, the first diaper having the first decorative portion and the marked portion and the second diaper having the second decorative portion and the marked portion improve decorativeness and attract the user's attention, and the visibility of the marked portion can be improved. The first and second decorative portions are portions having designs other than the marked portion, and include not only a design having symbols or patterns but also background color consisting of only color.

Hitherto, the present invention has been described in detail using the above-described embodiment, but it is obvious to those skilled in the art that the present invention is not limited to the embodiments described in the present specification. The present invention can be carried out as a correction and modification aspect within the gist and scope of the present invention determined by the recitation in claims. Therefore, the description of the present specification is intended for exemplary description and does not mean to limit the present invention by any means.

The entire contents of Japanese Patent Application No. 2019-094865, filed May 20, 2019, are incorporated into the present specification by reference.

### INDUSTRIAL APPLICABILITY

The present invention can provide a pet diaper which can be put on a proper position and can inhibit the leakage of excrement.

### REFERENCE SIGNS LIST

1, 1X, 1Y, 1Z, 1V: pet diaper
2: main-body portion
10 topsheet
20: backsheet
30: absorbent core
40: joining portion
13: side stretchable member (stretchable member)
50: marked portion
51: first marked portion
52: second marked portion
55: stretching marked portion
56: boundary marked portion
57: end edge marked portion
58: region marked portion
R50: space region
R60: hole region
R100: product region
W: waistline direction
Z: cross direction

1. A pet diaper having a waistline direction to be arranged along a waistline direction of a pet and a cross direction orthogonal to the waistline direction, the pet diaper comprising:
a main-body portion having a topsheet, a backsheet, and an absorbent core arranged between the topsheet and the backsheet; and
a joining portion that joins a backsheet-side surface of the main-body portion and a topsheet-side surface of the main-body portion;
wherein the absorbent core is arranged away from two end edges of the main-body portion toward an inner side in the waistline direction, and
the pet diaper has a marked portion as a guide showing where the pet diaper is to be put on.

2. The pet diaper according to claim 1,
wherein the marked portion is configured to be visible from the topsheet side of the pet diaper.

3. The pet diaper according to claim 1,
wherein the marked portion is configured to be visible from the backsheet side of the pet diaper.

4. The pet diaper according to claim 1,
wherein the marked portion has a waist center marked portion that indicates a center of the absorbent core in the waistline direction.

5. The pet diaper according to claim 1,
wherein the marked portion is a plurality of marked portions arranged in the waistline direction by being spaced apart from each other.

6. The pet diaper according to claim 5,
wherein the marked portion has a pair of waist side marked portions arranged on two sides facing each other across a center of the absorbent core in the waistline direction.

7. The pet diaper according to claim 6,
wherein one of the pair of waist side marked portions is arranged in a region that overlaps one end portion of the absorbent core in the waistline direction, and the other is arranged in a region that overlaps the other end portion of the absorbent core in the waistline direction.

8. The pet diaper according to claim 6,
wherein one of the pair of waist side marked portions is arranged away from one end edge of the absorbent core in the waistline direction toward an outer side in the waistline direction, and the other is arranged away from the other end edge of the absorbent core in the waistline direction toward the outer side in the waistline direction.

9. The pet diaper according to claim 1,
wherein a stretchable member that stretches and contracts in at least one of the waistline direction and the cross direction is arranged in the main-body portion, and
the marked portion has a stretching marked portion arranged in a region that overlaps the stretchable member.

10. The pet diaper according to claim 1,
wherein the absorbent core is arranged away from the two end edges of the main-body portion toward an inner side in the cross direction, and
the marked portion has a cross center marked portion that indicates a center of the absorbent core in the cross direction.

11. The pet diaper according to claim 1,
wherein the marked portion is a plurality of marked portions arranged in the cross direction by being spaced apart from each other.

12. The pet diaper according to claim 11,
wherein the marked portion has a pair of cross side marked portions that are arranged in the cross direction on two sides facing each other across a center of the absorbent core in the cross direction.

13. The pet diaper according to claim 12,
wherein one of the pair of cross side marked portions is arranged in a region that overlaps one end portion of the absorbent core in the cross direction, and the other is arranged in a region that overlaps the other end portion of the absorbent core in the cross direction.

14. The pet diaper according to claim 12,
wherein one of the pair of cross side marked portions is arranged away from one end edge of the absorbent core in the cross direction toward an outer side in the cross direction, and the other is arranged away from the other end edge of the absorbent core in the cross direction toward the outer side in the cross direction.

15. The pet diaper according to claim 1,
wherein a first decorative portion and a second decorative portion different from the first decorative portion in design are printed on the backsheet, and
the marked portion has a boundary marked portion configured with a boundary between the first decorative portion and the second decorative portion.

16. The pet diaper according to claim 1,
wherein the marked portion has an end edge marked portion that indicates an outer end edge of the main-body portion in the cross direction.

17. The pet diaper according to claim 1,
wherein the joining portion is provided in a first end portion of body that is one end portion of the main-body portion in the waistline direction and configured to join the first end portion of body to a second end portion of body that is the other end portion of the main-body portion in the waistline direction,
the main-body portion has an indicator that is arranged between the backsheet and the absorbent core and changes color when coming into contact with moisture,
the indicator extends in the waistline direction, and
in the waistline direction, a length of the indicator is 50% or more of a length of a product region that extends between an end edge of the main-body portion on a side of the second end portion of body and the joining portion.

18. The pet diaper according to claim 17,
wherein the main-body portion has four equally divided regions determined by dividing the product region in quarters in the waistline direction,
the four equally divided regions have a first product region that is closest to a side of the first end portion of body and a second product region that is closest to the side of the second end portion of body, and
the indicator is arranged in at least one of the first product region and the second product region.

19. The pet diaper according to claim 18,
wherein the indicator is arranged in both the first product region and the second product region.

20. (canceled)

21. The pet diaper according to claim 17,
wherein in the waistline direction, the length of the indicator is 80% or more of the length of the absorbent core.

22-27. (canceled)
